(19) United States
(12) Patent Application Publication
(10) Pub. No.: US 2010/0143994 A1
(43) Pub. Date: Jun. 10, 2010

(54) PROCESS FOR FERMENTIVE PREPARATION OF ALCOHOLS AND RECOVERY OF PRODUCT

(75) Inventors: Jeanine M. Erdner-Tindall, Salem, NJ (US); Keith W. Hutchenson, Lincoln University, PA (US); Mark Brandon Shiflett, Landenberg, PA (US); Ranjan Patnaik, Hockessin, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E. I. DU PONT DE MEMOURS AND COMPANY

(21) Appl. No.: 12/328,308

(22) Filed: Dec. 4, 2008

(51) Int. Cl. C12P 7/16

(52) U.S. Cl. 435/160

(57) ABSTRACT

This invention relates to a process for recovering an alcohol from a fermentation broth using liquid-liquid extraction, wherein at least one ionic liquid is used as the extractive solvent.

### TECHNICAL FIELD

This invention relates to a process for preparing an alcohol in, and recovering such product from, a fermentation medium.

### BACKGROUND

Production of chemicals from renewable resources is typically achieved by fermentation of sugars derived from biomass using either naturally isolated microorganisms or genetically modified miroorganisms. The economic viability of such processes, especially for commodity products such as organic acids, amino acids, vitamins, and more recently biofuels such as ethanol, butanol or higher alcohols, is dependent on high volumetric productivity and yield of the fermentation process. In many cases, the accumulation of the desired product at high concentration in the fermentation medium inhibits the metabolism of the microorganisms, which slows or effectively stops the fermentation process. One approach for alleviating this limitation is to genetically modify the production organism to be more tolerant to the inhibitory product or compounds. An alternative engineering approach is the continuous removal during fermentation of the product or the inhibitory compound, using in-situ product removal (ISPR), with the result that the effective concentration in the reactor is maintained below the threshold toxicity level tolerated by the microorganism.

Liquid-liquid extraction (LLE) is an ISPR technique in which a desired compound (such as a fermentation product) is preferentially extracted from a first liquid phase into a second immiscible liquid phase that can easily be separated from the first liquid phase. The desired compound can then be recovered from the second immiscible phase.

Pfruender et al [J. Biotechnology (2006) 124:182-190] disclose that resting cell suspensions of Saccharomyces cerevisiae can carry out the biocatalytic synthesis of (S)-4-chloro-3-hydroxybutanoate in the presence of specific ionic liquids, such as 1-n-butyl-3-methylimidazolium hexafluorophosphate and 1-n-butyl-3-methylimidazolium bis(trifluoromethanesulfonyl)imide. The assays were carried out in a potassium phosphate buffer supplemented with sodium chloride.

A need nevertheless remains for processes for preparing various compounds by fermentation in which the product can be recovered using ISPR techniques.

### SUMMARY

In one embodiment, there is provided herein a process for preparing an alcohol in a fermentation broth in a fermentor by (a) providing a liquid fermentation broth that is comprised of a carbohydrate substrate, nutrients and water in which an alcohol is produced by the growth of a microorganism; (b) contacting at least one ionic liquid with the fermentation broth, or a portion thereof, to form from the resulting mixture an ionic liquid phase and an aqueous phase wherein the alcohol, or a portion thereof, is more soluble in the ionic liquid phase than the aqueous phase; and (c) separating the alcohol-rich ionic liquid phase from the aqueous phase; and, optionally, recovering the alcohol from the ionic liquid phase.

An ionic liquid is well suited to serve as a solvent to separate an alcohol product from a fermentation broth because ionic liquids will generally have no measurable vapor pressure, and because of the availability of ionic liquids that have high solubility for the alcohol product, are themselves immiscible with the aqueous fermentation broth, and have little to no toxicity to the microorganism.

### BRIEF DESCRIPTION OF THE DRAWINGS

FIG. 1 illustrates a process for recovering ethanol from fermentation broth.

FIG. 2 shows tables of Glucose Uptake Index (GUI) and ethanol production profiles in shake flask cultures in the presence of selected IL's.

### DETAILED DESCRIPTION

This invention relates to a process for preparing an alcohol in an aqueous fermentation broth wherein the alcohol product is recovered by contacting one or more ionic liquids with the fermentation broth. Product recovery proceeds generally in the form of a liquid-liquid extraction based on a relatively higher extent of solubility of the alcohol product in an ionic liquid than in the aqueous fermentation broth, and on the relative lack of miscibility between the ionic liquid and the aqueous fermentation broth. The alcohol obtained from the fermentation, and thus recovered as the product, may be any one or more of ethanol, 1-butanol, 2-butanol, isobutanol (2-methyl-1-propanol) or tertiary butanol (2-methyl-2-propanol).

Ethanol and butanol are both important industrial commodity chemicals with a variety of applications. Both have been used as fuels or fuel additives, but the potential of butanol is this respect is particularly significant. Although only a four-carbon alcohol, butanol has an energy content similar to that of gasoline and can be blended with any fossil fuel. Butanol is favored as a fuel or fuel additive as it yields only CO₂ and little or no SOₓ or NOₓ when burned in the standard internal combustion engine, and it causes a limited amount of corrosion.

Processes by which alcohols are made by fermentation are well known (see, for example, Bailey, Biochemical Engineering Fundamentals, Second Edition, McGraw Hill, New York, 1986). Fermentation is the enzyme-catalyzed, energy-yielding pathway in cells by which sugar molecules are metabolically broken down by microorganisms in a series of oxidation and reduction reactions. The fermentation can begin with a sugar such as glucose, or can begin, for example, with starch, which is a polymeric form of glucose with a high molecular weight. Before the cell of a microorganism can carry out an alcoholic fermentation, the starch must be broken down to its constituent glucose units, and this may be performed, for example, by the enzyme amylase (diastase), which may in some cases be produced upon germination of grain from which the starch was obtained.

In the series of reactions in which sugar molecules are broken down, some of the energy that is released is stored for future use in the high energy chemical bonds of adenosine triphosphate (ATP). Hydrolysis of the energy-rich pyrophosphate bonds of ATP provides energy used to drive the biosynthetic reactions necessary for cell growth and multiplication. In these reactions, energy is derived from the partial oxidation of organic compounds using organic intermediates as electron donors and electron acceptors, and using NAD as an oxidizing agent, and NADH₂ is a reducing agent.

For example, a six carbon sugar such as glucose may be broken down into two molecules of the three-carbon organic acid pyruvic acid (or its ionized form pyruvate) coupled with the transfer of chemical energy to the synthesis of ATP. The pyruvate may then be reduced to an alcohol. When, for example, ethanol is produced from pyruvate, a yeast may irreversibly decarboxylate pyruvate with the aid of pyruvate decarboxylase (2-oxo acid carboxylase) to yield acetaldehyde. Alcohol dehydrogenase (NAD oxidoreductase) then catalyzes the reduction of acetaldehyde to ethanol.

Sugars suitable for fermentation herein as a carbohydrate substrate may be obtained from a variety of crop and waste materials such as sugarcane juice, molasses, sugar beet, corn steep liquor, cassava, sweet potatoes, sweet sorghum, Jerusalem artichoke, primary clarifier sludge, newsprint, cardboard, cotton linters, rice straw, rice hulls and corn stillage. For cellulosic biomasss such as agricultural residues, forestry residues, waste paper and yard waste, the cellulose and hemicellulose in these materials, which are long chain polymers made up of sugar molecules, can be treated with dilute acid hydrolysis at a temp of about 240° C. to hydrolyze the cellulose and hemicellulose to break down the molecules into smaller fractions that can be readily fermented. Alternatively, cellulose enzymes can be used to hydrolyze the cellulose to glucose for direct fermentation.

The actual sugar molecules that are subjected to fermentation herein typically include without limitation the hexose sugars of D-glucose, D-fructose and D-mannose, and frequently also sucrose, maltose, maltotriose, raffinose and D-galactose. Some strains of usable microorganisms, however, do not metabolize L-sugars or pentoses at a commercially viable rate.

In addition to the carbohydrate substrate, the culture medium (growth medium) as used in a process hereof will contain various nutrients. Included among the nutrients typically used in this fermentation process are nitrogen, minerals and trace elements, and vitamins, as well as other growth factors.

Suitable growth factors include vitamins, purines, pyrimidines, nucleotides, nucleosides, amino acids, fatty acids, sterols and polyamines. Nitrogen may be obtained from sources such as gaseous ammonia; ammonium salts such as ammonium sulfate or diammonium hydrogen phosphate; nitrates; urea; organic forms of nitrogen such as mixtures of peptides and amino acids (which may in turn be obtained from hydrolysed plant protein material such as corn steep liquor, casein hydrolysate, soybean meal, barley malt, corn gluten meal, linseed meal, whey powder, beet and cane molasses, rice and wheat meal, and yeast extract); and peptones, which are protein hydrolysates derived from meat, casein, gelatin, keratin, peanuts, soybean meal, cottonseeds, and sunflower seeds.

Suitable minerals and elements typically include phosphorus [e.g. (NH₄)₂HPO₄], potassium (e.g. KCl), magnesium, sulfur (e.g. MgSO₄.7H₂O) sodium, chlorine, cobalt, nickel (e.g. NiCl₂), iron (e.g. FeCl₂.H₂O), zinc (e.g. ZnCl₂), manganese, calcium (e.g. CaCl₂), copper (e.g. CuSO₄.5H₂O), and molybdenum (e.g. Na₂MoO₄). Suitable vitamins typically include riboflavin, nicotinic acid, pantothenic acid, folic acid, choline, inositol, biotin, pyroxidine, and thiamin.

In a process hereof, the microorganisms that are used to obtain an alcohol as a result of their growth in the presence of the carbohydrate substrate should have high selectivity, low accumulation of byproducts, high alcohol yield, high fermentation rate, good tolerance toward both increased alcohol and substrate concentrations, good tolerance toward the extracting solvent, and good tolerance toward lower pH values. It has been found that microorganisms suitable for use herein include without limitation a Saccharomyces, a recombinant Saccharomyces, a Lactobacillus, or a recombinant Lactobacillus. For example, the various known strains of Saccharomyces include S. carlsbergensis, S. diastaticus, S. cerevisiea, S. bayanus, S. uvarum, S. pastorianus and S. exiguous; and the various known strains of Lactobacillus include Lactobacillus fermentum, Lactobacillus zeae, and Lactobacillus rhamnosus.

Saccharomyces can be grown in a fermentor as discussed by Kosaric et al in Ullmann's Encyclopedia of Industrial Chemistry, Sixth Edition, Volume 12, pages 398-473 (Wiley-VCH Verlag GmbH & Co. KDaA, Weinheim, Germany). As reported therein, S. cerevisiae can be grown in a YPD growth medium containing carbon; and nutrients such as nitrogen, phosphorus, sulfur, hydrogen, minor quantities of postassium, magnesium, calcium, trace minerals and organic growth factors; where a YPD medium contains yeast extract, peptone and dextrose.

A recombinant strain of Saccharomyces or of Lactobacillus for use herein will be made for the purpose of improving cellular activities by manipulation of enzymatic transport and regulatory functions of the cell with the use of recombinant DNA technology. The objective is the expression of new genes in various host cells, and the amplification of endogenous enzymes and deletion of genes or modulation of enzyme activities. The host microorganisms used are those that are capable of being genetically altered to produce the necessary enzymes to form a metabolic pathway for the production of ethanol or a butanol. The microorganism may naturally possess some of the enzymes in the pathway, but will not be able to complete the job until it has been genetically altered, and exogenous genes will thus be added to complete a metabolic pathway. The manner of genetic alteration may use any combination of known genetic engineering techniques such as mutation or addition of foreign DNA. Foreign DNA may be introduced into the microorganism by any conventional technique such as conjugation, transformation, transduction or electroporation.

A gene may be added to a cell by way of a vector. The vector may be in the form of a plasmid, cosmid or virus which is compatible with the cell's DNA and any resident plasmids. Generally, vectors either integrate into the recipient microorganism's DNA or the vector has an origin of replication to stably maintain the vector throughout many microbial generations. The origin of replication may code for either stringent or non-stringent replication. To express the gene(s), a structural gene is generally placed downstream from a promotor region on the DNA. The promotor must be recognized by the recipient microorganism. In addition to the promotor, one may include, delete or modify regulatory sequences to either increase expression or to control expression. Expression may be controlled by an inducer or a repressor so that the recipient microorganism expresses the gene(s) only when desired.

For example, U.S. Pat. No. 5,916,787 (which is by this reference incorporated in its entirety as a part hereof for all purposes) discloses the genetic transformation of a Gram-positive bacteria such as Lactobacillus with genes that confer upon the bacteria the capability of producing useful levels of ethanol, and in particular discloses the transformation of the host with heterologous genes such as those taken from Z. mobilis, that encode the pyruvate decarboxylase and alcohol dehydrogenase enzymes resulting in the production of enzymes that redirect the metabolism of the transformed host such that ethanol is produced as a primary fermentation product of the host.

It is reported by Senthilkumar and Gunasekaran in 64 Journal of Scientific and Industrial Research 845˜853 (November 2005) that pyruvate decarboxylase and alcohol dehydrogenase genes from Z. mobilis were transformed into Lactobacillus casei by means of an expression vector based in part on Lactococcus lactis, which resulted in the production of ethanol in fermentation at a rate better than the parental Z. mobilis. See, also, Gold et al, 33 Curr. Microbiology 256˜260 (1996).

More recently, U.S. Patent Publication No. 2007/0092957, in paragraph 3 through paragraph 290, including Examples 17 through 19, describes the synthesis of isobutanol using a recombinant S. cerevisiae. Example 21 describes the synthesis of isobutanol using a recombinant Lactobacillus. In one embodiment, U.S. 2007/0092957 provides a recombinant S. cerevisiae strain containing at least one DNA molecule that encodes a polypeptide that catalyzes a substrate to product conversion selected from the group consisting of (i) pyruvate to acetolactate; (ii) acetolactate to 2,3-dihydroisovalerate; (iii) 2,3-dihydroisovalerate to α-ketoisovalerate; (iv) α-ketoisovalerate to isobutyraldehyde; and (v) isobutyraldehyde to isobutanol. In an additional embodiment, U.S. 2007/0092957 provides a method for producing isobutanol using said S. cerevisiae strain. Additional S. cerevisiae hosts and methods for producing isobutanol are also described. In another embodiment, U.S. 2007/0092957 provides a recombinant Lactobacillus plantarum strain containing at least one DNA molecule that encodes a polypeptide that catalyzes a substrate to isobutanol product conversion according to the same pathway described above.

U.S. Patent Publication No. 2007/0259410 (paragraph 8 through paragraph 309), and U.S. Patent Publication No. 2007/0292927 (paragraph 8 through paragraph 349), describe a method for synthesizing 2-butanol using a recombinant S. cerevisiae, and describe a method for synthesizing 2-butanol using a recombinant Lactobacillus. In one embodiment, U.S. Patent Publication No. 2007/0259410 provides a recombinant S. cerevisiae strain containing at least one DNA molecule that encodes a polypeptide that catalyzes a substrate to product conversion selected from the group consisting of (i) pyruvate to alpha-acetolactate; (ii) alpha-acetolactate to acetoin; (iii) acetoin to 3-amino-2-butanol; (iv) 3-amino-2-butanol to 3-amino-2-butanol phosphate; (v) 3-amino-2-butanol phosphate to 2-butanone; and (vi) 2-butanone to 2-butanol. In another embodiment, U.S. 2007/0259410 provides a recombinant Lactobacillus strain containing at least one DNA molecule that encodes a polypeptide that catalyzes a substrate to 2-butanol product conversion according to the same pathway described above.

In another embodiment, U.S. Patent Publication No. 2007/0292927 provides a recombinant S. cerevisiae strain containing at least one DNA molecule that encodes a polypeptide that catalyzes a substrate to product conversion selected from the group consisting of (i) pyruvate to alpha-acetolactate; (ii) alpha-acetolactate to acetoin; (iii) acetoin to 2,3-butanediol; (iv) 2,3-butanediol to 2-butanone; and (v) 2-butanone to 2-butanol. Additional S. cerevisiae hosts and methods for producing isobutanol are also described. In another embodiment, U.S. 2007/0292927 provides a recombinant Lactobacillus strain containing at least one DNA molecule that encodes a polypeptide that catalyzes a substrate to 2-butanol product conversion according to the same pathway described above.

In another embodiment, WO 2007/041269 describes the synthesis of 1-butanol using a recombinant S. cerevisiae. In one embodiment, WO 2007/041269 provides a recombinant S. cerevisiae strain containing at least one DNA molecule that encodes a polypeptide that catalyzes a substrate to product conversion selected from the group consisting of (i) acetyl-GoA to acetoacetyl-GoA; (ii) acetoacetyl-GoA to 3-hydroxybutyryl-GoA; (iii) 3-hydroxybutyryl-GoA to crotonyl-GoA; (iv) crotonyl-GoA to butyryl-GoA; (v) butyryl-GoA to butyraldehyde; and (vi) butyraldehyde tol-butanol. Additional S. cerevisiae hosts and methods for producing 1-butanol are also described. In another embodiment, WO 2007/041269 provides a recombinant Lactobacillus strain containing at least one DNA molecule that encodes a polypeptide that catalyzes a substrate to 1-butanol product conversion according to the same pathway described above.

U.S. Pat. No. 5,916,787; U.S. Patent Publication No. 2007/0092957; U.S. Patent Publication No. 2007/0259410; and U.S. Patent Publication No. 2007/0292927 is each by this reference incorporated in its entirety as a part hereof for all purposes.

In a process hereof, the culture medium and microorganisms are contacted in a fermentation broth, which is an aqueous solution or slurry of those materials formed by the addition of water. The type of process used to conduct the fermentation may be either batch, fed batch (in which sterile culture medium is added continuously or periodically to the inoculated fermentation batch, and the volume of the fermentation broth increases with each addition of medium), or continuous in which sterile medium is fed continuously into the fermenter and the fermented product is continuously withdrawn so the fermentation volume remains unchanged). Preferably, the process is a continuous process.

Good contacting between the various components of the reaction mixture in the broth may be obtained with rotating impellers, an airlift (which has separate riser and downcomer channels to circulate the liquid), or a trickle bed (which has a gas flow up from the bottom). Where there is a possibility that the fermentation could be damaged by excessive heat during sterilization, sterilization may optionally be performed by passage of nutrients and other components through hydrophilic polymer filters.

The fermentation process may be controlled by measuring and monitoring relevant conditions and variables, which may include one or more of the following: temperature, pressure, gas flow rate, liquid inlet and outlet flow rates, culture level, culture volume, culture weight, culture viscosity, agitation power, agitation speed, foaming, dissolved oxygen concentration, dissolved oxygen tension, dissolved CO₂ concentration, redox potential, pH, conductivity, ionic strength, dilution rate, carbohydrate concentration, total protein concentration, vitamin concentration, nucleic acid concentration, total cell count, viable cell count, biomass concentration, cell size and age, and doubling time.

Measurement of reaction conditions and variables may be performed using analytical methods such as high performance liquid chromatography, nuclear magnetic resonance, flow cytometry, or fluorometry. In one embodiment, for example, flow injection analysis, mass spectrometry or gas chromatography may be used to measure biomass concentration, substrate uptake rate or product formation rate, but the latter two are often preferred. Biomass concentration may also be measured (in the case of bacteria) by measuring turbidity, or (in the case of a fungus) by measuring dry weight, but in situ methods based on optical, calorimetric, acoustic, fluorimetric, or capacitance readings are also suitable.

In other embodiments, on-line measurement of ethanol concentration can be made using a sensor that consists of an immobilized cell membrane of Gluconobacter oxydans in calcium alginate containing pyrrolo-quinoline quinine, coated with a nitrocellulose layer. In yet other embodiments, using freeze/quench methods, the concentration of cofactors such as ATP and NADH, ADP and AMP may be measured on samples withdrawn from the fermentation reactor. In addition, metabolic flux analysis may be performed on the primary intracellular fluxes by applying a stoichiometric model and mass balances to the substrate uptake rate and metabolite secretion rate using ¹³C-enriched carbon sources and measurement of the fractional enrichment of ¹³C in the intracellular metabolites.

The fermentation may be run generally at a temperature in the range of about 0° C. to about 50° C., or in the range of about 25° C. to about 45° C., or in the range of about 30° C. to about 40° C. The pH is often somewhat acidic, with optimum pH typically in the range of about 4.5 to about 6.5, although there is usually tolerance to lower pHs such as below 3 or even below 2. The microorganism is usually added to the fermentation medium in an amount of about 100 or more colony forming units per mL of medium, or in an amount of about 10-20 million cells per mL of medium.

Where desired, the microorganisms themselves may be removed from the fermentation broth by flocculation, centrifugation and/or filtration after they have fulfilled their metabolic role. This may be done before of after the broth is contacted with an ionic liquid as described herein. Where further desired, the microorganism cells may be recycled to the broth for the purpose of increasing productivity. Recycling of microorganism cells creates a high biomass concentration at the beginning of the process, which reduces the time for the conversion of substrate to product.

For example, where it is desired to use a Saccharomyces yeast as the microorganism, a flocculating type of yeast may be of particular interest. These cells can readily be concentrated and separated without the use of mechanical devices such as a centrifuge or settlers. A highly flocculating yeast such as Saccharomyces diastaticus has been found, at the end of fermentation and after agitation has stopped, to be able to settle rapidly in the bioreactor in about one minute. A tower reactor is convenient for internal settling of flocculating yeast cells. The clear supernatant formed by the settling, which is the alcohol-containing liquid broth, may then be subjected to the separation methods hereof in the reactor, or decanted and separated by such methods outside the reactor. Next, fresh culture medium may be added to the bioreactor, which starts a new fermentation batch. These cycles can be repeated ten times or more without loss in productivity and cell viability. High alcohol productivity is achieved with a very short fermentation time.

In other embodiments, a high concentration of microorganism cells may be obtained in the fermentor by various cell immobilization techniques, e.g. by entrapment in a gel matrix, covalent binding to surfaces of various support materials, or adsorption on a support. These systems do not require agitation. The immobilized cells are retained in the reactor, and cell separation devices and recycle are thus not needed. High dilution rates without cell washout can be achieved. Immobilized cells can be used in fixed- and fluidized-bed reactors. In these cases, the substrate solution flows continuously through the reactor, and the immobilized cells convert available sugar to alcohol. Calcium alginate [9005-35-0] can be used to entrap the cells.

In a process of this invention, at a preselected point in time during the fermentative production of an alcohol, intermittently according to a preselected schedule, or continuously during the process of fermentation, the fermentation broth is subjected to liquid-liquid extraction, either in the fermentor or in an external vessel, to remove the alcohol product [a technique commonly referred to as “in-situ product removal” (ISPR)].

ISPR is performed in a process hereof using liquid-liquid extraction (LLE) methods, and ionic liquids are well suited to serve as the extractant in such a fermentation-coupled LLE-ISPR since they typically have no measureable vapor pressure and little solubility in the aqueous phase. Ionic liquids are organic compounds that are liquid at a temperature of less than about 100° C., and preferably at room temperature (approximately 25° C.). They differ from most salts in that they have very low melting points, and they generally tend to be liquid over a wide temperature range. They also generally tend to not be soluble in non-polar hydrocarbons; to be immiscible with water (depending on the anion); and to be highly ionizing (but have a low dielectric strength). Ionic liquids have essentially no vapor pressure, most are air and water stable, and they can either be neutral, acidic or basic.

A cation or anion of an ionic liquid useful herein can in principle be any cation or anion such that they together form an organic salt that is liquid at or below about 100° C. The properties of an ionic liquid can, however, be tailored by varying the identity of the cation and/or anion. For example, the acidity of an ionic liquid can be adjusted by varying the molar equivalents and type and combinations of Lewis acids used. This provides flexibility in not only modulating their biocompatibility properties in respect of a variety of microorganisms, but also enables the use of techniques such as distillation and centrifugation to separate a product from the ionic liquid. ISPR as practiced herein is particularly desirable in a fermentation process because it can enhance biomass growth by keeping the alcohol product at a concentration that is not toxic to the microorganism, and yet do so by use of an ionic liquid that is itself not toxic to the microorganism.

Liquid-liquid extraction is a process for separating components in solution by their distribution between two immiscible liquid phases. Liquid-liquid extraction involves the transfer of mass from one liquid phase into a second immiscible liquid phase, and is carried out using an extractant (i.e. solvent). An “extractant” or “solvent” for use in liquid-liquid extraction is an immiscible liquid that, when added to a mixture, interacts with the components in the mixture in such a way that one or more, and preferably one, of the components in the mixture is more soluble in the extractant than one or more other components, and is more soluble in the extractant than in the mixture, thereby causing separation of the more soluble component or components from the mixture. The liquid phase that remains after separation of the more soluble component or components is the “extract”. In a process hereof, one or more ionic liquids is used as the extractant.

The transfer of mass from one liquid phase into a separate immiscible phase by liquid-liquid extraction can be carried out in several ways as may be illustrated by the manner of operation of known LLE processes, which include the recovery of acetic acid from water using ethyl ether or ethyl acetate as the extractant [as described in Brown, Chem. Engr. Prog. (1963) 59:65], and the recovery of phenolics from water with methyl isobutyl ketone as the extractant [as described by Scheibel in “Liquid-Liquid Extraction”, Chapter 3 of Separation and Purification, 3^{rd} Ed. (Perry and Weissburg), John Wiley & Sons, Inc. (1978)]. LLE is also discussed by Robbins et al in “Liquid-Liquid Extraction Operations and Equipment” in Perry's Chemical Engineers' Handbook, 7^{th} Ed. (McGraw-Hill, 1997, Section 15).

Ethanol or butanol can be separated by liquid-liquid extraction in either continuous or batch mode using a single equilibrium (i.e. theoretical) stage, or using multiple stages. An equilibrium (theoretical) stage is a device that allows intimate mixing of a feed (e.g. a fermentation broth) with an immiscible liquid such that concentrations approach equilibrium, followed by physical separation of the two immiscible liquid phases. A single stage device can be a separatory funnel, or an agitated vessel, which allows for intimate mixing of the feed with the immiscible extractant. Following intimate mixing, one or both of the liquid phases can be recovered by decantation, for example.

Multiple stage devices can be crosscurrent or countercurrent devices. In a multiple stage device, the feed enters a first equilibrium stage and is contacted with an extractant. The two liquid phases are mixed, with droplets of one phase suspended in the second phase, and then the two phases are separated, and the raffinate from the first stage is contacted with additional extractant, and the separation process is repeated. “Raffinate” is the liquid phase that is left from the feed after the feed is contacted with the extractant, and one or more components are partially or completely removed. The process of 1) contacting the raffinate with extractant, 2) allowing for equilibrium concentrations to be approached, and 3) separating the liquid phases is repeated until a sufficient amount of ethanol or butanol is removed from the feed. The number of equilibrium stages will depend on the desired purity, as well as the solubility of ethanol or butanol in the extractant and the flow rates of the fermentation broth and extractant.

In a crosscurrent system (or device), the feed is initially contacted with extractant in a first equilibrium stage. The raffinate from this stage then cascades down through one or more additional stages. At each stage the raffinate is contacted with fresh extractant, and further removal of ethanol or butanol from the raffinate is achieved. In a crosscurrent system (or device), the extractant enters at the stage farthest from the feed, and the two phases pass countercurrently to one another.

Equipment used for liquid-liquid extraction can be classified as “stagewise” or “continuous (differential) contact” equipment, and equipment that is typically used is further discussed by Robbins, supra. Stagewise equipment is also referred to as “mixer-settlers”. Mixing the liquids occurs by contacting the feed with the extractant, and the resultant dispersion is settled as the two phases separate. Mixing can occur with the use of baffles or impellers, and the separation process may be carried out in batch fashion or with continuous flow. Settlers can be simple gravity settlers, such as decanters, or can be cyclones or centrifuges, which enhance the rate of settling.

Continuous contact equipment is typically arranged for multistage countercurrent contact of the immiscible liquids, without repeated separation of the liquids from each other between stages. Instead, the liquids remain in continuous contact throughout their passage through the equipment. Countercurrent flow is maintained by the difference in densities of the liquids and either the force of gravity (vertical towers) or centrifugal force (centrifugal extractors). Gravity-operated extractors can be classified as spray towers, packed towers or perforated-plate (sieve-plate) towers. Gravity-operated towers also include towers with rotating stirrers and pulsed towers as is known in the art.

Any of the equipment described above can be used for the separation of ethanol or butanol from a fermentation broth using an ionic liquid as the extractant. In one preferred embodiment, the separation is carried out using a vertical tower with perforated plates.

In a process hereof, at least one ionic liquid is contacted with the fermentation broth to form from the resulting mixture an ionic liquid phase and an aqueous phase wherein the alcohol product, or a portion thereof, is more soluble in the ionic liquid phase than the aqueous phase. The alcohol-rich ionic liquid phase is then separated from the aqueous phase; and the alcohol product is optionally recovered from the ionic liquid phase.

The alcohol-rich ionic liquid phase can be separated from the aqueous phase derived from the fermentation broth by any suitable means such as decantation or centrifugation. The product alcohol can be recovered from the ionic liquid phase using standard distillation techniques such as are discussed in Seader et al (“Distillation”, in Perry's Chemical Engineer's Handbook, 7^{th} Edition, Section 13, 1997, McGraw-Hill, New York).

The aqueous phase, i.e. the residual fermentation broth, can remain in or be recycled back to the fermentor to continue the alcohol production process. Make-up medium components, such as glucose or other carbon sources and nutrients, can be added to the fermentor as necessary; in addition, a portion of the reduced-ethanol fermentation broth stream that is recycled to the fermentor can be purged as needed.

In other embodiments of a process hereof, during or after separation of the ionic liquid phase from the aqueous phase, production of the alcohol product in the fermentation broth continues. Separation of the two phases may be conducted in the fermenter, or may be conducted in a vessel external to the fermenter. When separation occurs in an external vessel, after separation of the aqueous phase from the ionic liquid phase, the aqueous phase or a portion thereof may be returned to the fermenter. After alcohol recovery, the ionic liquid may also be recycled to the fermentation broth.

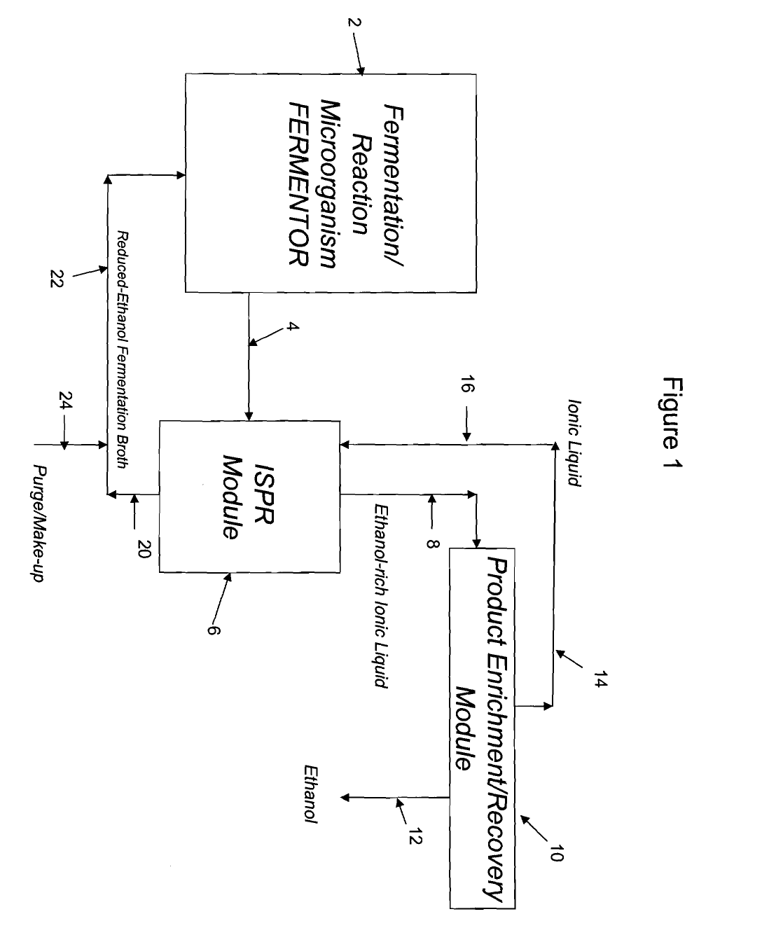

One embodiment of a process hereof is shown in FIG. 1. In FIG. 1, there is a shown a block diagram of an apparatus for recovering ethanol from fermentation broth. A culture of microorganism is grown in fermenter 2 until a desired concentration of ethanol in the fermentation broth is achieved. Typically, the target ethanol concentration is chosen so that the rate of ethanol production by the microorganism is not significantly inhibited by accumulation of product. A stream 4 comprising at least one portion of the fermentation broth is fed into ISPR Module 6 which is typically a mixing tank/decanter or a Karr column, wherein the portion of the fermentation broth is contacted with an ionic liquid. The volume ratio of ionic liquid to fermentation broth can be from about 10:1 to about 1:1. Stream 20, the fermentation broth that is reduced in ethanol content, exits the ISPR Module. One or more purge/make-up streams 24 are fed into stream 20 to form stream 22, which is pumped (pump not shown) back into fermenter 2. Stream 8, the ethanol-rich ionic liquid phase is fed into Product Recovery Module 10, which can be a distillation column having a sufficient number of theoretical stages to cause separation of the ethanol from the ionic liquid. Ethanol is recovered from Product Module 10 as stream 12. The ionic liquid exits Product Module 10 as stream 14, where it can be recycled to ISPR Module 6 as stream 16. The contents of the FIG. 1, and the embodiment shown therein, apply equally to a process in which butanol is the fermentation product rather than ethanol.

Other methods of product recovery from a fermentation broth that are applicable herein are discussed in U.S. Pat. No. 4,865,973, which is by this reference incorporated in its entirety as a part hereof for all purposes.

In another embodiment, the process hereof further includes a step of mixing the ethanol or butanol product recovered with a motor fuel such as gasoline.

Numerous ionic liquids are suitable for use as the extractant in the fermentation coupled LLE-ISPR process hereof. Representative examples of typical ionic liquids are described in sources such as J. Chem. Tech. Biotechnol., 68:351-356 (1997); Chem. Ind., 68:249-263 (1996); J. Phys. Condensed Matter, 5: (supp 34B):B99-B106 (1993); Chemical and Engineering News, Mar. 30, 1998, 32-37; J. Mater. Chem., 8:2627-2636 (1998); Chem. Rev., 99:2071-2084 (1999); and US 2004/0133058 (which is by this reference incorporated as a part hereof for all purposes). In one embodiment hereof, a library, i.e. a combinatorial library, of ionic liquids may be prepared, for example, by preparing various alkyl derivatives of a particular cation (such as the quaternary ammonium cation), and varying the associated anions.

Mixtures of ionic liquids may also be useful for achieving proper extraction of ethanol and/or butanol from a fermentation broth where, for example, differing levels of partition coefficient and toxicity may be balanced between a selection of two or more ionic liquids.

Many ionic liquids are formed by reacting a nitrogen-containing heterocyclic ring, preferably a heteroaromatic ring, with an alkylating agent (for example, an alkyl halide) to form a quaternary ammonium salt, and performing ion exchange or other suitable reactions with various Lewis acids or their conjugate bases to form the ionic liquid. Examples of suitable heteroaromatic rings include substituted pyridines, imidazole, substituted imidazole, pyrrole and substituted pyrroles. These rings can be alkylated with virtually any straight, branched or cyclic C₁₋₂₀ alkyl group, but preferably, the alkyl groups are C₁₋₁₆ groups, since groups larger than this may produce low melting solids rather than ionic liquids. Various triarylphosphines, thioethers and cyclic and non-cyclic quaternary ammonium salts may also been used for this purpose. Counterions that may be used include chloroaluminate, bromoaluminate, gallium chloride, tetrafluoroborate, tetrachloroborate, hexafluorophosphate, nitrate, trifluoromethane sulfonate, methylsulfonate, p-toluenesulfonate, hexafluoroantimonate, hexafluoroarsenate, tetrachloroaluminate, tetrabromoaluminate, perchlorate, hydroxide anion, copper dichloride anion, iron trichloride anion, zinc trichloride anion, as well as various lanthanum, potassium, lithium, nickel, cobalt, manganese, and other metal-containing anions.

Ionic liquids may also be synthesized by salt metathesis, by an acid-base neutralization reaction or by quaternizing a selected nitrogen-containing compound; or they may be obtained commercially from several companies such as Merck (Darmstadt, Germany) or BASF (Mount Olive, N.J.). Methods of synthesizing specific ionic liquids useful in a process hereof are set forth below.

### Group I Ionic Liquids

One group of ionic liquids suitable for use in a process hereof may include a cation selected from the group consisting of imidazolium, pyridinium, phosphonium or pyrrolidinium.

A pyridinium cation may be represented by the structure of the following formula:

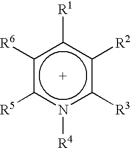

An imidazolium cation may be represented by the structure of the following formula:

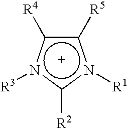

A phosphonium cation may be represented by the structure of the following formula:

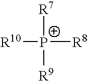

A pyrrolidinium cation may be represented by the structure of the following formula:

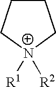

wherein R¹ through R⁶ is each independently —CH₃, —C₂H₅, or C₃ to C₆ straight-chain or branched alkane or alkene group, and R⁷through R¹⁰ is each independently —CH₃, —C₂H₅, or a C₃ to C₁₅ straight-chain or branched alkane or alkene group.

Group I ionic liquids may further include an anion selected from the group consisting of tris(pentafluoroethyl)trifluorophosphate (FAP), 1,1,2-trifluoro-2-(perfluoroethoxy)ethanesulfonate (TPES), 1,1,2-trifluoro-2-(perfluoromethoxy)ethanesulfonate (TTES), bis(pentafluoroethylsulfonyl)imide (BEI) bis(trifluoromethylsulfonyl)imide (Tf₂N), tetrafluoroborate (BF₄), hexafluorophosphate (PF₆), 1,1,2,3,3,3-hexafluoropropanesulfonate (HFPS), and 2-(1,2,2,2-tetrafluoroethoxy)-1,1,2,2-tetrafluoroethanesulfonate bis(pentafluoroethylsulfonyl)imide (FS).

In one embodiment of the Group I ionic liquids, the cation is selected from the group consisting of 1-hexyl-3-methylimidazolium (HMIM), tetradecyl(tri-n-hexyl)phosphonium (6,6,6,14-P), 1-butyl-3-methylimidazolium (BMIM), 1-ethyl-3-methylimidazolium (EMIM), 3-methyl-1-propylpyridinium (PMPy), and 1-butyl-1-methylpyrrolidinium (BMP).

In a more specific embodiment of the Group I ionic liquids, the cation may be 1-ethyl-3-methylimidazolium, and the anion may be selected from the group consisting of (FAP), (TPES), (TTES), (BEI), (Tf₂N), (BF₄), (PF₆), (HFPS) or (FS). In another embodiment, the cation may be 1-butyl-3-methylimidazolium, and the anion may be selected from the group consisting of (FAP), (TPES), (TTES), (BEI), (Tf₂N), (BF₄), (PF₆), (HFPS) or (FS). In yet another embodiment, the cation may be 1-hexyl-3-methylimidazolium, and the anion may be selected from the group consisting of (FAP), (TPES), (TTES), (BEI), (Tf₂N), (BF₄), (PF₆), (HFPS) or (FS). In yet another embodiment, the cation may be 3-methyl-1-propylpyridinium, and the anion may be selected from the group consisting of (FAP), (TPES), (TTES), (BEI), (Tf₂N), (BF₄), (PF₆), (HFPS) or (FS). In yet another embodiment, the cation may be tetradecyl(tri-n-hexyl-phosphonium, and the anion may be selected from the group consisting of (FAP), (TPES), (TTES), (BEI), (Tf₂N), (BF₄), (PF₆), (HFPS) or (FS). In yet another embodiment, the cation may be 1-butyl-1-methylpyrrolidinium, and the anion may be selected from the group consisting of (FAP), (TPES), (TTES), (BEI), (Tf₂N), (BF₄), (PF₆), (HFPS) or (FS).

In an even more specific embodiment, the Group I ionic liquids may be selected from the group consisting of 1-hexyl-3-methylimidazolium tris(pentafluoroethyl)trifluorophosphate, tetradecyl(tri-n-hexyl)phosphonium 1,1,2-trifluoro-2-(perfluoroethoxy)ethanesulfonate, 1-butyl-3-methylimidazolium tetrafluoroborate, 1-butyl-3-methylimidazolium 1,1,2-trifluoro-2-(perfluoroethoxy)ethanesulfonate, 1-ethyl-3-methylimidazolium bis(pentafluoroethylsulfonyl)imide, 1-ethyl-3-methylimidazolium bis(trifluoromethylsulfonyl)imide, 1-butyl-3-methylimidazolium hexafluorophosphate, 1-butyl-3-methylimidazolium bis(trifluoromethylsulfonyl)imide, 1-hexyl-3-methylimidazolium bis(trifluoromethylsulfonyl)imide, 1-butyl-3-methylimidazolium 1,1,2-trifluoro-2-(trifluoromethoxy)ethanesulfonate, 1-butyl-3-methylimidazolium 1,1,2,3,3,3-hexafluoropropanesulfonate, 3-methyl-1-propylpyridinium bis(trifluoromethylsulfonyl)imide, 1-butyl-3-methylimidazolium 2-(1,2,2,2-tetrafluoroethoxy)-1,1,2,2-tetrafluoroethanesulfonate, 1-butyl-3-methylimidazolium 1,1,2,3,3,3-hexafluoropropanesulfonate, and 1-butyl-3-methylpyrrolidinium bis(trifluoromethylsulfonyl)imide.

Group I ionic liquids may be made by various methods of synthesis, as follows:

### Synthesis of Anions

### Synthesis of potassium-1,1,2-trifluoro-2-(perfluoroethoxy)ethanesulfonate (TPES-K)

A 1-gallon Hastelloy® C276 reaction vessel was charged with a solution of potassium sulfite hydrate (88 g, 0.56 mol), potassium metabisulfite (340 g, 1.53 mol) and deionized water (2000 ml). The vessel was cooled to 7 degrees C., evacuated to 0.05 MPa, and purged with nitrogen. The evacuate/purge cycle was repeated two more times. To the vessel was then added perfluoro(ethylvinyl ether) (PEVE, 600 g, 2.78 mol), and it was heated to 125 degrees C. at which time the inside pressure was 2.31 MPa. The reaction temperature was maintained at 125 degrees C. for 10 hr. The pressure dropped to 0.26 MPa at which point the vessel was vented and cooled to 25 degrees C. The crude reaction product was a white crystalline precipitate with a colorless aqueous layer (pH=7) above it.

The ¹⁹F NMR spectrum of the white solid showed pure desired product, while the spectrum of the aqueous layer showed a small but detectable amount of a fluorinated impurity. The desired isomer is less soluble in water so it precipitated in isomerically pure form.

The product slurry was suction filtered through a fritted glass funnel, and the wet cake was dried in a vacuum oven (60 degrees C., 0.01 MPa) for 48 hr. The product was obtained as off-white crystals (904 g, 97% yield).

¹⁹F NMR (D₂O) δ −86.5 (s, 3F); −89.2, −91.3 (subsplit ABq, J_{FF}=147 Hz, 2F);

−119.3, −121.2 (subsplit ABq, J_{FF}=258 Hz, 2F); −144.3 (dm, J_{FH}=53 Hz, 1F).

¹H NMR (D₂O) δ 6.7 (dm, J_{FH}=53 Hz, 1H).

Mp (DSC) 263 degrees C.

Analytical calculation for C₄HO₄F₈SK: C, 14.3: H, 0.3 Experimental results: C, 14.1: H, 0.3.

TGA (air): 10% wt. loss @ 359 degrees C., 50% wt. loss @ 367 degrees C.

TGA (N₂): 10% wt. loss @ 362 degrees C., 50% wt. loss @ 374 degrees C.

### Synthesis of potassium-1,1,2-trifluoro-2-(trifluoromethoxy)ethanesulfonate (TTES-K)

A 1-gallon Hastelloy® C276 reaction vessel was charged with a solution of potassium sulfite hydrate (114 g, 0.72 mol), potassium metabisulfite (440 g, 1.98 mol) and deionized water (2000 ml). The pH of this solution was 5.8. The vessel was cooled to −35 degrees C., evacuated to 0.08 MPa, and purged with nitrogen. The evacuate/purge cycle was repeated two more times. To the vessel was then added perfluoro(methylvinyl ether) (PMVE, 600 g, 3.61 mol) and it was heated to 125 degrees C. at which time the inside pressure was 3.29 MPa. The reaction temperature was maintained at 125 degrees C. for 6 hr. The pressure dropped to 0.27 MPa at which point the vessel was vented and cooled to 25 degrees C. Once cooled, a white crystalline precipitate of the desired product formed leaving a colorless clear aqueous solution above it (pH=7).

The ¹⁹F NMR spectrum of the white solid showed pure desired product, while the spectrum of the aqueous layer showed a small but detectable amount of a fluorinated impurity.

The solution was suction filtered through a fritted glass funnel for 6 hr to remove most of the water. The wet cake was then dried in a vacuum oven at 0.01 MPa and 50 degrees C. for 48 hr. This gave 854 g (83% yield) of a white powder. The final product was isomerically pure (by ¹⁹F and ¹H NMR) since the undesired isomer remained in the water during filtration.

¹⁹F NMR (D₂O) δ −59.9 (d, J_{FH}=4 Hz, 3F); −119.6, −120.2 (subsplit ABq, J=260 Hz, 2F); −144.9 (dm, J_{FH}=53 Hz, 1F).

¹H NMR (D₂O) δ . . . 6.6 (dm, J_{FH}=53 Hz, 1H).

% Water by Karl-Fisher titration: 71 ppm.

Analytical calculation for C₃HF₆SO₄K: C, 12.6: H, 0.4: N, 0.0 Experimental results: C, 12.6: H, 0.0: N, 0.1.

Mp (DSC) 257 degrees C.

TGA (air): 10% wt. loss @ 343 degrees C., 50% wt. loss @ 358 degrees C.

TGA (N₂): 10% wt. loss @ 341 degrees C., 50% wt. loss @ 357 degrees C.

### Synthesis of sodium 1,1,2,3,3,3-hexafluoropropanesulfonate (HFPS-Na)

A 1-gallon Hastelloy® C reaction vessel was charged with a solution of anhydrous sodium sulfite (25 g, 0.20 mol), sodium bisulfite 73 g, (0.70 mol) and of deionized water (400 ml). The pH of this solution was 5.7. The vessel was cooled to 4 degrees C., evacuated to 0.08 MPa, and then charged with hexafluoropropene (HFP, 120 g, 0.8 mol, 0.43 MPa). The vessel was heated with agitation to 120 degrees C. and kept there for 3 hr. The pressure rose to a maximum of 1.83 MPa and then dropped down to 0.27 MPa within 30 minutes. At the end, the vessel was cooled and the remaining HFP was vented, and the reactor was purged with nitrogen. The final solution had a pH of 7.3.

The water was removed in vacuo on a rotary evaporator to produce a wet solid. The solid was then placed in a vacuum oven (0.02 MPa, 140 degrees C., 48 hr) to produce 219 g of white solid which contained approximately 1 wt % water. The theoretical mass of total solids was 217 g. The crude HFPS-Na can be further purified and isolated by extraction with reagent grade acetone, filtration, and drying.

¹⁹F NMR (D₂O) δ −74.5 (m, 3F); −113.1, −120.4 (ABq, J=264 Hz, 2F); −211.6 (dm, 1F).

¹H NMR (D₂O) δ . . . 5.8 (dm, J_{FH}=43 Hz, 1H).

MP (DSC) 126 degrees C.

TGA (air): 10% wt. loss @ 326 degrees C., 50% wt. loss @ 446 degrees C.

TGA (N₂): 10% wt. loss @ 322 degrees C., 50% wt. loss @ 449 degrees C.

### Synthesis of Group I Ionic Liquids

### Synthesis of Tetradecyl(tri-n-hexyl)phosphonium 1,1,2-trifluoro-2-(perfluoroethoxy)ethanesulfonate([6.6.6.14]P-TPES)

To a 500 ml round bottomed flask was added acetone (Spectroscopic grade, 50 ml) and ionic liquid tetradecyl(tri-n-hexyl)phosphonium chloride (Cyphos® IL 101, 33.7 g). The mixture was magnetically stirred until it was one phase. In a separate 1 liter flask, potassium 1,1,2-trifluoro-2-(perfluoroethoxy)ethanesulfonate (TPES-K, 21.6 g) was dissolved in acetone (400 ml). These solutions were combined and stirred under positive N₂ pressure at 26 degrees C. for 12 hr producing a white precipitate of KCl. The precipitate was removed by suction filtration, and the acetone was removed in vacuo on a rotovap to produce the crude product as a cloudy oil (48 g). Chloroform (100 ml) was added, and the solution was washed once with deionized water (50 ml). It was then dried over magnesium sulfate and reduced in vacuo first on a rotovap and then on a high vacuum line (8 Pa, 24 degrees C.) for 8 hr to yield the final product as a slightly yellow oil (28 g, 56% yield).

¹⁹F NMR (DMSO-d₆) δ −86.1 (s, 3F); −88.4, −90.3 (subsplit ABq, J_{FF}=147 Hz, 2F); −121.4, −122.4 (subsplit ABq, J_{FF}=258 Hz, 2F); −143.0 (dm, J_{FH}=53 Hz, 1F).

¹H NMR (DMSO-d₆) δ 0.9 (m, 12H); 1.2 (m, 16H); 1.3 (m, 16H); 1.4 (m, 8H); 1.5 (m, 8H); 2.2 (m, 8H); 6.3 (dm, J_{FH}=54 Hz, 1H).

% Water by Karl-Fisher titration: 0.11.

Analytical calculation for C₃₆H₆₉F₈O₄PS: C, 55.4: H, 8.9: N, 0.0. Experimental Results: C, 55.2: H, 8.2: N, 0.1.

TGA (air): 10% wt. loss @ 311 degrees C., 50% wt. loss @ 339 degrees C.

TGA (N₂): 10% wt. loss @ 315 degrees C., 50% wt. loss @ 343 degrees C.

### Synthesis of 1-butyl-3-methylimidazolium 1,1,2,3,3,3-hexafluoropropanesulfonate (Bmim-HFPS)

1-Butyl-3-methylimidazolium chloride (Bmim-Cl, 50.0 g) and high purity dry acetone (>99.5%, 500 ml) were combined in a 1 liter flask and warmed to reflux with magnetic stirring until the solid all dissolved. At room temperature in a separate 1 liter flask, potassium-1,1,2,3,3,3-hexafluoropropanesulfonte (HFPS-K) was dissolved in high purity dry acetone (550 ml). These two solutions were combined at room temperature and allowed to stir magnetically for 12 hr under positive nitrogen pressure. The stirring was stopped, and the KCl precipitate was allowed to settle. This solid was removed by suction filtration through a fritted glass funnel with a celite pad. The acetone was removed in vacuo to give a yellow oil. The oil was further purified by diluting with high purity acetone (100 ml) and stirring with decolorizing carbon (5 g). The mixture was suction filtered and the acetone removed in vacuo to give a colorless oil. This was further dried at 4 Pa and 25 degrees C. for 2 hr to provide 68.6 g of product.

¹⁹F NMR (DMSO-d₆) δ −73.8 (. . . s, 3F); −114.5, −121.0 (ABq, J=258 Hz, 2F); −210.6 (m, J=42 Hz, 1F).

¹H NMR (DMSO-d₆) δ 0.9 (t, J=7.4 Hz, 3H); 1.3 (m, 2H); 1.8 (m, 2H); 3.9 (s, 3H); 4.2 (t, J=7 Hz, 2H); 5.8 (dm, J=42 Hz, 1H); 7.7 (s, 1H); 7.8 (s, 1H); 9.1 (s, 1H).

% Water by Karl-Fisher titration: 0.12%.

Analytical calculation for C₉H₁₂F₆N₂O₃S: C, 35.7: H, 4.4: N, 7.6. Experimental Results: C, 34.7: H, 3.8: N, 7.2.

TGA (air): 10% wt. loss @ 340 degrees C., 50% wt. loss @ 367 degrees C.

TGA (N₂): 10% wt. loss @ 335 degrees C., 50% wt. loss @ 361 degrees C.

Extractable chloride by ion chromatography: 27 ppm.

### Synthesis of 1-butyl-3-methylimidazolium 1,1,2-trifluoro-2-(trifluoromethoxy)ethanesulfonate

1-Butyl-3-methylimidazolium chloride (Bmim-Cl, 10.0 g) and deionized water (15 ml) were combined at room temperature in a 200 ml flask. At room temperature in a separate 200 ml flask, potassium 1,1,2-trifluoro-2-(trifluoromethoxy)ethanesulfonate (TTES-K, 16.4 g) was dissolved in deionized water (90 ml). These two solutions were combined at room temperature and allowed to stir magnetically for 30 min. under positive nitrogen pressure to give a biphasic mixture with the desired ionic liquid as the bottom phase. The layers were separated, and the aqueous phase was extracted with 2×50 ml portions of methylene chloride. The combined organic layers were dried over magnesium sulfate and concentrated in vacuo. The colorless oil product was dried at for 4 hr at 5 Pa and 25 degrees C. to afford 15.0 g of product.

¹⁹F NMR (DMSO-d₆) δ −56.8 (d, J_{FH}=4 Hz, 3F); −119.5, −119.9 (subsplit ABq, J=260 Hz, 2F); −142.2 (dm, J_{FH}=53 Hz, 1F).

¹H NMR (DMSO-d₆) δ 0.9 (t, J=7.4 Hz, 3H); 1.3 (m, 2H); 1.8 (m, 2H); 3.9 (s, 3H); 4.2 (t, J=7.0 Hz, 2H); 6.5 (dt, J=53 Hz, J=7 Hz, 1H); 7.7 (s, 1H); 7.8 (s, 1H); 9.1 (s, 1H).

% Water by Karl-Fisher titration: 613 ppm.

Analytical calculation for C₁₁H₁₆F₆N₂O₄S: C, 34.2: H, 4.2: N, 7.3. Experimental Results: C, 34.0: H, 4.0: N, 7.1.

TGA (air): 10% wt. loss @ 328 degrees C., 50% wt. loss @ 354 degrees C.

TGA (N₂): 10% wt. loss @ 324 degrees C., 50% wt. loss @ 351 degrees C.

Extractable chloride by ion chromatography: <2 ppm.

### Synthesis of 1-butyl-3-methylimidazolium 1,1,2-trifluoro-2-(perfluoroethoxy)ethanesulfonate (Bmim-TPES)

1-Butyl-3-methylimidazolium chloride (Bmim-Cl, 7.8 g) and dry acetone (150 ml) were combined at room temperature in a 500 ml flask. At room temperature in a separate 200 ml flask, potassium 1,1,2-trifluoro-2-(perfluoroethoxy)ethanesulfonate (TPES-K, 15.0 g) was dissolved in dry acetone (300 ml). These two solutions were combined and allowed to stir magnetically for 12 hr under positive nitrogen pressure. The KCl precipitate was then allowed to settle leaving a colorless solution above it. The reaction mixture was filtered once through a celite/acetone pad and again through a fritted glass funnel to remove the KCl. The acetone was removed in vacuo first on a rotovap and then on a high vacuum line (4 Pa, 25 degrees C.) for 2 hr. Residual KCl was still precipitating out of the solution, so methylene chloride (50 ml) was added to the crude product which was then washed with deionized water (2×50 ml). The solution was dried over magnesium sulfate, and the solvent was removed in vacuo to give the product as a viscous light yellow oil (12.0 g, 62% yield).

¹⁹F NMR (CD₃CN) δ −85.8 (s, 3F); −87.9, −90.1 (subsplit ABq, J_{FF}=147 Hz, 2F); −120.6, −122.4 (subsplit ABq, J_{FF}=258 Hz, 2F); −142.2 (dm, J_{FH}=53 Hz, 1F).

¹H NMR (CD₃CN) δ 1.0. (t, J=7.4 Hz, 3H); 1.4 (m, 2H); 1.8 (m, 2H); 3.9 (s, 3H); 4.2 (t, J=7.0 Hz, 2H); 6.5 (dm, J=53 Hz, 1H); 7.4 (s, 1H); 7.5 (s, 1H); 8.6 (s, 1H).

% Water by Karl-Fisher titration: 0.461.

Analytical calculation for C₁₂H₁₆F₈N₂O₄S: C, 33.0: H, 3.7. Experimental Results: C, 32.0: H, 3.6.

TGA (air): 10% wt. loss @ 334 degrees C., 50% wt. loss @ 353 degrees C.

TGA (N₂): 10% wt. loss @ 330 degrees C., 50% wt. loss @ 365 degrees C.

### Group II Ionic Liquids

A second group of ionic liquids suitable for use in a process hereof may include a cation selected from the group consisting of pyrrolidinium and imidazolium.

A pyrolidinium cation is derived from a pyrrolidone compound such as a pyrrolidine-2-one (2-pyrrolidone) compound, and may be represented by the structure of the following formula:

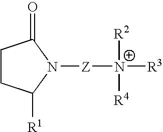

wherein Z is —(CH₂)ₙ— wherein n is an integer from 2 to 12, R¹ is —CH₃, and R², R³ and R⁴ are each independently —CH₃, —CH₂CH₃ or a C₃ to C₆ straight-chain or branched monovalent alkyl group.

An imidazolium cation may be represented by the structure of the following formula:

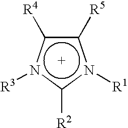

wherein R¹ through R⁶ are each independently —CH₃, —C₂H₅, or a C₃ to C₆ straight-chain or branched alkane or alkene group, and R⁷ through R¹⁰ are each independently —CH₃, —C₂H₅, or a C₃ to C₁₅ straight-chain or branched alkane or alkene group.

Group II ionic liquids may further include an anion selected from the group consisting of levulinate (Lev), bis(trifluoromethane)sulfonamide (Tf₂N) and hexafluoropropanesulfonate (HFPS).

In one embodiment of the Group II ionic liquids, the cation is selected from the group consisting of 1-ethyl-3-methylimidazolium (EMIM), 1-butyl-3-methylimidazolium (BMIM), 1-(N,N,N-dimethyl(methylethoxy)aminoethyl)-5-methyl pyrrolidone-2-one (MeDMMEAP), 1-(N,N,N-dimethyl(ethylethoxy)aminoethyl)-5-methyl pyrrolidone-2-one (DMEEAP), 1-(N,N,N-dimethylpropylaminoethyl)-5-methyl pyrrolidone-2-one (MeDMPAP), or 1-(N,N,N-dimethyl(hydoxyethyl)aminoethyl)-5-methyl pyrrolidone-2-one (MeDHEAP).

In a more specific embodiment of the Group II ionic liquids, the cation may be 1-ethyl-3-methylimidazolium, and the anion may be selected from the group consisting of (Lev), (HFPS), or (Tf₂N). In another embodiment, the cation may be 1-butyl-3-methylimidazolium, and the anion may be selected from the group consisting of (Lev), (HFPS) or (Tf₂N). In yet another embodiment, the cation may be 1-(N,N,N-dimethyl(methylethoxy)aminoethyl)-5-methyl pyrrolidone-2-one, and the anion may be selected from the group consisting of (Lev), (HFPS) or (Tf₂N). In yet another embodiment, the cation may be 1-(N,N,N-dimethyl(ethylethoxy)aminoethyl)-5-methyl pyrrolidone-2-one, and the anion may be selected from the group consisting of (Lev), (HFPS) or (Tf₂N). In yet another embodiment, the cation may be 1-(N,N,N-dimethylpropylaminoethyl)-5-methyl pyrrolidone-2-one, and the anion may be selected from the group consisting of (Lev), (HFPS) or (Tf₂N). In yet another embodiment, the cation may be 1-(N,N,N-dimethyl(hydoxyethyl)aminoethyl)-5-methyl pyrrolidone-2-one, and the anion may be selected from the group consisting of (Lev), (HFPS) or (Tf₂N) as the anion.

In an even more specific embodiment, the Group II ionic liquids may be selected from the group consisting of 1-ethyl-3-methylimidazolium levulinate, 1-(N,N,N-dimethyl(ethylethoxy)aminoethyl)-5-methyl pyrrolidone-2-one hexafluoropropanesulfonate, 1-(N,N,N-dimethyl(ethylethoxy)aminoethyl)-5-methyl pyrrolidone-2-one bis(trifluoromethane)sulfonamide, 1-(N,N,N-dimethyl(methylethoxy)aminoethyl)-5-methyl pyrrolidone-2-one hexafluoropropanesulfonate, 1-butyl-3-methylimidazolium levulinate, 1-(N,N,N-dimethylpropylaminoethyl)-5-methyl pyrrolidone-2-one bis(trifluoromethane)sulfonamide, 1-(N,N,N-dimethyl(methylethoxy)aminoethyl)-5-methyl pyrrolidone-2-one bis(trifluoromethane)sulfonamide, or 1-(N,N,N-dimethyl(hydoxyethyl)aminoethyl)-5-methyl pyrrolidone-2-one bis(trifluoromethane)sulfonamide.

Group II ionic liquids may be made by various methods of synthesis, as follows:

### Materials.

The following materials were used in the synthesis of Group II ionic liquids. The commercial reagents and solvents
acetonitrile (CAS Registry No. 75-05-8, 99.8% purity),
dichloromethane (CAS Registry No. 75-09-2, 99.5% purity),
diethyl ether (CAS Registry No. 60-29-7, 99% purity),
2-chloroethyl ethyl ether (CAS Registry No. 628-34-2, 98% purity, Fluka product),
ethyl levulinate (CAS Registry No. 539-88-8, 99% purity),
ethyl acetate (CAS Registry No. 141-78-6, 99.8% purity),
levulinic acid (CAS Registry No. 123-76-2, 98% purity),
silver (I) oxide (CAS Registry No. 20667-12-3, 99% purity),
bis(trifluoromethane)sulfonimide (CAS Registry No 82113-65-3, 97% purity, Fluka product), and
N,N-dimethylethylenediamine (CAS Registry No. 108-00-9, 98.0% purity, Fluka product)
   were obtained from Sigma-Aldrich Chemical Company (Milwaukee, Wis., USA) and used as received without further purification. Potassium hexafluoropropanesulfonate (CAS Registry No. 905298-79-5, 95+% purity) was prepared according to a method set forth in U.S. Patent Publication 2006/0276671. ESCAT® 142 catalyst (5 wt % palladium on activated carbon) was obtained from Engelhard (now BASF Catalysts, Iselin N.J.).

### Synthesis of sodium 1,1,2,3,3,3-hexafluoropropanesulfonate (HFPS-Na)

A 1-gallon Hastelloy® C reaction vessel was charged with a solution of anhydrous sodium sulfite (25 g, 0.20 mol), sodium bisulfite 73 g, (0.70 mol) and of deionized water (400 ml). The pH of this solution was 5.7. The vessel was cooled to 4 degrees C., evacuated to 0.08 MPa, and then charged with hexafluoropropene (HFP, 120 g, 0.8 mol, 0.43 MPa). The vessel was heated with agitation to 120 degrees C. and kept there for 3 hr. The pressure rose to a maximum of 1.83 MPa and then dropped down to 0.27 MPa within 30 minutes. At the end, the vessel was cooled and the remaining HFP was vented, and the reactor was purged with nitrogen. The final solution had a pH of 7.3.

The water was removed in vacuo on a rotary evaporator to produce a wet solid. The solid was then placed in a vacuum oven (0.02 MPa, 140 degrees C., 48 hr) to produce 219 g of white solid which contained approximately 1 wt % water. The theoretical mass of total solids was 217 g. The crude HFPS-Na can be further purified and isolated by extraction with reagent grade acetone, filtration, and drying.

¹⁹F NMR (D₂O) δ −74.5 (m, 3F); −113.1, −120.4 (ABq, J=264 Hz, 2F); −211.6 (dm, 1F).

¹H NMR (D₂O) δ 5.8 (dm, J_{FH}=43 Hz, 1H).

Mp (DSC) 126 degrees C.

TGA (air): 10% wt. loss @ 326 degrees C., 50% wt. loss @ 446 degrees C.

TGA (N₂): 10% wt. loss @ 322 degrees C., 50% wt. loss @ 449 degrees C.

### Synthesis of Group II Ionic Liquids

### Synthesis of 1-(2-(dimethylamino)ethyl)-5-methylpyrrolidin-2-one (MeDMAP)

1-(2-(dimethylamino)ethyl)-5-methylpyrrolidin-2-one (MeDMAP), C₉H₁₈N₂O, with a molecular weight of 170.25 g mol⁻¹ and structure as shown in Formula I:

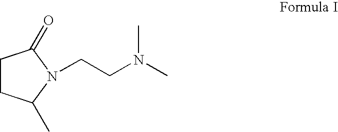

was prepared as follows via the cyclic reductive amination of ethyl levulinate with N,N-dimethylethylenediamine (as described in U.S. Pat. No. 7,157,588).

To a 600-mL Hastelloy® C-276 autoclave reactor (Parr Model 2302 HC) equipped with a gas entrainment turbine impellor and electrical heating mantle was added 150.0 g (1.04 mol) ethyl levulinate, 192.6 g (2.18 mol) N,N-dimethylethylenediamine, and 7.5 g ESCAT® 142 5% Pd/C catalyst. The reactor was purged first with nitrogen and then hydrogen, and then pressurized with 50 psig (0.4 MPa) hydrogen and stirred at 600 rpm while heating the reaction mixture to 150° C. On reaching this reaction temperature, the reactor was further pressurized to 1000 psig (7.0 MPa) with hydrogen and maintained at this pressure by adding additional hydrogen as required for the duration of the reaction. After 6 hours at these conditions, the reactor was cooled and vented, and the liquid reaction mixture was recovered for product isolation. The crude mixture was filtered through a glass frit via aspirator vacuum to remove the catalyst followed by removal of byproduct ethanol and unreacted N,N-dimethylethylenediamine in vacuo. The remaining contents were fractionally distilled with a 20-cm Vigreaux column under high vacuum (˜0.05 mmHg) to give 136.5 g water-white product at 85° C. in 77% isolated yield. Product purity was >99% as determined by GC/MS (HP-6890 equipped with MSD).

### Synthesis of 1-(N,N,N-dimethyl(ethylethoxy)aminoethyl)-5-methyl-pyrrolidine-2-one bis(trifluoromethane)sulfonamide ([MeDMEEAP] [TF₂N])

1-(N,N,N-dimethyl(ethylethoxy)aminoethyl)-5-methyl-pyrrolidine-2-one bis(trifluoromethane)-sulfonamide ([MeDMEEAP] [TF₂N]), C₁₅H₂₇N₃O₆F₆S₂, with a molecular weight of 523.51 g mol⁻¹ and structure as shown in Formula II was prepared as follows:

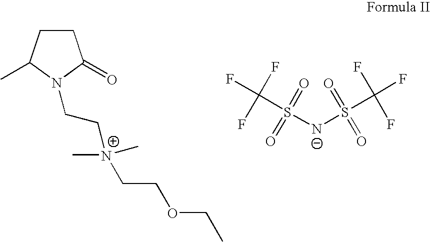

1-(2-(dimethylamino)ethyl)-5-methylpyrrolidin-2-one (MeDMAP), C₉H₁₈N₂O, with a molecular weight of 170.25 g mol⁻¹ and a purity of >99% by GC/MS, was used as prepared above. To a two-neck 100-mL round bottom flask equipped with a nitrogen-purged reflux condenser was added 24.27 g (0.143 moles) MeDMAP, 30.40 g (0.280 moles) 2-chloroethyl ethyl ether, and 17.81 g acetonitrile as reaction solvent. The condenser was cooled by a recirculating bath filled with a 50 wt % mixture of water and propylene glycol maintained at approximately 16° C. The reaction mixture was heated to 85° C. under reflux and nitrogen purge with a temperature-controlled oil bath. This reaction temperature was maintained for 120 hrs, at which time the conversion of the MeDMP was about 94.4% by ¹H NMR spectroscopy. The reaction mixture was then thermally quenched and extracted with multiple diethyl ether and ethyl acetate washes to remove starting materials and to purify the intermediate product. The solvents were removed in vacuo with a rotary evaporator, and the intermediate product was then dried under high vacuum (approximately 10⁻⁶ torr) using a turbomolecular pump and heating the material to about 70-80° C. overnight. The resulting intermediate product of this reaction, 1-(N,N,N-dimethyl(ethylethoxy)aminoethyl)-5-methyl-pyrrolidine-2-one chloride ([MeDMEEAP] [Cl]), C₁₃H₂₇N₂O₂Cl, with a molecular weight of 278.82 g mol⁻¹, was determined to have a final purity of about 95.1% by ¹H NMR spectroscopy.

In a 500-mL round bottom flask, 11.50 g (0.0413 mol) of this [MeDMEEAP] [Cl] intermediate was dissolved in approximately 150 mL of purified water and then mixed with 12.81 g (0.0456 mol) bis(trifluoromethane)sulfonimide dissolved in approximately 150 mL water. After stirring the reaction solution overnight at room temperature, the resulting IL was purified by extracting the resulting hydrochloric acid and the excess bis(trifluoromethane)-sulfonamide with multiple water washes of about 15 mL each while keeping the IL product partitioned in an organic phase with dichloromethane. Water was removed from the filtrate in vacuo with a rotary evaporator, then the product was dried under high vacuum (approximately 10⁻⁵ torr) using a turbomolecular pump and heating the material to about 70° C. overnight. The resulting [MeDMEEAP] [Tf₂N] product purity was estimated to be about 95% by ¹H NMR spectroscopy.

### Synthesis of 1-(N,N,N-dimethyl(ethylethoxy)aminoethyl)-5-methyl-pyrrolidine-2-one hexafluoropropanesulfonate ([MeDMEEAP] [HFPS])

1-(N,N,N-dimethyl(ethylethoxy)aminoethyl)-5-methyl-pyrrolidine-2-one hexafluoropropanesulfonate ([MeDMEEAP] [HFPS]), C₁₅H₂₆N₂O₅F₆S, with a molecular weight of 460.43 g mol⁻¹ and structure as shown in Formula III, was prepared as follows:

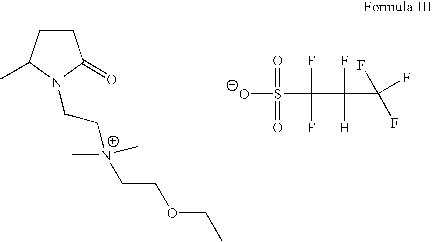

1-(N,N,N-dimethyl(ethylethoxy)aminoethyl)-5-methyl-pyrrolidine-2-one chloride ([MeDMEEAP] [Cl]), C₁₃H₂₇N₂O₂Cl, with a molecular weight of 278.82 g mol⁻¹ and final purity of about 95.1% by ¹H NMR spectroscopy, was used as prepared above. In a 500-mL round bottom flask, 11.28 g (0.0405 mol) of this 95% [MeDMEEAP] [Cl] intermediate was dissolved in approximately 150 mL of acetone and then mixed in a substoichiometric amount of 10.51 g (0.0389 mol) potassium hexafluoropropanesulfonate. After stirring the reaction solution overnight at room temperature, the IL product was filtered to remove the resulting potassium chloride crystals. The filtrate was allowed to set for about a week, and additional potassium chloride crystals formed and were removed by filtration. The solvent was removed in vacuo with a rotary evaporator, and then the product was dried under high vacuum (approximately 10⁻⁵ torr) using a turbomolecular pump and heating the material to about 70° C. overnight. The final purity of the resulting [MeDMEEAP] [HFPS] product was approximately 98% by ¹H NMR spectroscopy.

### Synthesis of 1-(N,N,N-dimethyl(methylethoxy)aminoethyl)-5-methylpyrrolidin-2-one hexafluoropropanesulfonate ([MeDMMEAP] [HFPS])

1-(N,N,N-dimethyl(methylethoxy)aminoethyl)-5-methylpyrrolidin-2-one hexafluoropropanesulfonate ([MeDMMEAP] [HFPS]), C₁₅H₂₆N₂O₅F₆S, with a molecular weight of 460.43 g mol⁻¹ and structure as shown in Formula IV, was prepared as follows:

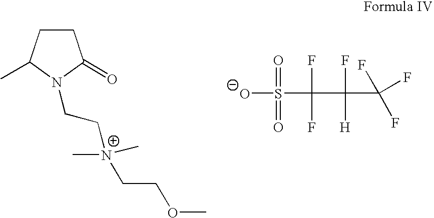

1-(N,N,N-dimethyl(methylethoxy)aminoethyl)-5-methylpyrrolidin-2-one chloride ([MeDMMEAP] [Cl]), C₁₂H₂₅N₂O₂Cl, with a molecular weight of 264.79 g mol⁻¹ and a purity of about 96.4% was used as prepared above. In a 500-mL round bottom flask, 12.94 g (0.0489 mol) of this 96.4% [MeDMMEAP] [Cl] intermediate was dissolved in approximately 100 mL of acetone, and then 14.59 g (0.0540 mol) potassium hexafluoropropanesulfonate was slowly added to this mixture. After stirring the reaction solution overnight at room temperature, the IL product was filtered with celite in a fritted funnel to remove the resulting potassium chloride crystals. The solvent was removed in vacuo with a rotary evaporator, and then the [MeDMMEAP] [HFPS] product was dissolved in dichloromethane and filtered through a column containing basic and neutral alumina. The dichloromethane solvent was removed in vacuo with a rotary evaporator, and then the product was dried under high vacuum (approximately 10⁻⁵ torr) using a turbomolecular pump and heating the material to about 70° C. overnight. The final purity of the resulting [MeDMMEAP] [HFPS] product was approximately 96% by ¹H NMR spectroscopy.

### Synthesis of 1-Butyl-3-methylimidazolium Levulinate ([BMIM) [Lev])

1-Butyl-3-methylimidazolium levulinate ([BMIM) [Lev]), with a structure as shown in Formula V, was prepared as follows:

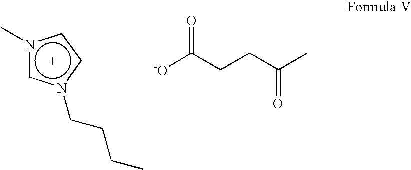

Water (300 mL) and silver (I) oxide (6.0 g, 0.026 mol) were charged to a 500-mL round bottom flask equipped with a magnetic stirbar. To the stirred dark black slurry, levulinic acid (5.8 g, 0.050 mol) was added. To the resulting dark brown stirred slurry, 1-butyl-3-methylimidazolium chloride ([BMIM] [Cl], 8.7 g, 0.050 mol) was then added. Upon addition of the ([BMIM] [Cl], the formation of a white precipitate (presumably AgCl) was evident. The reaction mixture was allowed to stir at ambient temperature for 16 hr, after which time the mixture appeared to be a tinted white slurry.

The resulting reaction mixture was filtered through an approximately 2-in pad of Celite filter aid (pre-wetted with water) on top of a fritted glass filter, and the filtrate containing the desired product was collected. The residual product in the pad of filter aid was rinsed from the filter aid with an additional three 30-mL portions of water and collected with the filtrate. The bulk of the water solvent was removed from the filtrate under vacuum with a rotary evaporator. The product was then further dried with a high-vacuum pump, leaving 10.6 g of product, which was analyzed by ¹H NMR spectroscopy in D₂O solvent.

### 1-Ethyl-3-methylimidazolium (EMIM) Levulinate

1-Ethyl-3-methylimidazolium levulinate ([EMIM) [Lev]), with a structure as shown in Formula VI, was prepared as follows:

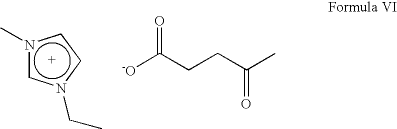

Water (300 mL) and silver (I) oxide (6.0 g, 0.026 mol) were charged to a 500-mL round bottom flask equipped with a magnetic stirbar. To the stirred dark black slurry, levulinic acid (6.0 g, 0.052 mol) was added. To the resulting dark brown stirred slurry, 1-ethyl-3-methylimidazolium chloride ([EMIM] [Cl]), 7.6 g, 0.052 mol) was then added. Upon addition of the [EMIM] [Cl], the formation of a white precipitate (believed to be AgCl) was evident. The reaction mixture was allowed to stir at ambient temperature for 16 hr, after which time the mixture appeared to be a tinted white slurry.

The resulting reaction mixture was filtered through an approximately 3-in pad of Celite filter aid (pre-wetted with water) on top of a fritted glass filter, and the filtrate containing the desired product was collected. The residual product in the pad of filter aid was rinsed from the filter aid with an additional three 30-mL portions of water and collected with the filtrate. The bulk of the water solvent was removed from the filtrate under vacuum with a rotary evaporator. The product was then further dried with a high-vacuum pump, leaving 8.3 g of product, which was analyzed by ¹H NMR spectroscopy in D₂O solvent.

In various other embodiments of this invention, an ionic liquid formed by selecting any of the individual cations described or disclosed herein, and by selecting any of the individual anions described or disclosed herein, may be used for the purpose of extracting an alcohol from a fermentation broth. Correspondingly, in yet other embodiments, a subgroup of ionic liquids formed by selecting (i) a subgroup of any size of cations, taken from the total group of cations described and disclosed herein in all the various different combinations of the individual members of that total group, and (ii) a subgroup of any size of anions, taken from the total group of anions described and disclosed herein in all the various different combinations of the individual members of that total group, may be used for the purpose of extracting an alcohol from a fermentation broth. In forming an ionic liquid, or a subgroup of ionic liquids, by making selections as aforesaid, the ionic liquid or subgroup will be identified by, and used in, the absence of the members of the group of cations and/or the group of anions that are omitted from the total group thereof to make the selection; and, if desirable, the selection may thus be made in terms of the members of one or both of the total groups that are omitted from use rather than the members of the group(s) that are included for use.

The advantageous attributes and effects of the processes hereof may be more fully appreciated from a series of examples as described below. The embodiments of these processes on which the examples are based are representative only, and the selection of those embodiments to illustrate the invention does not indicate that reactants, materials, conditions, regimes, protocols or techniques not described in these examples are not suitable for practicing these processes, or that subject matter not described in these examples is excluded from the scope of the appended claims and equivalents thereof.

### General Methods and Materials

Ionic liquids were obtained primarily from Fluka Chemika (and are also available from Sigma-Aldrich, St. Louis Mo.) with a purity of >97%. 1-Hexyl-3-methylimidazolium tris(pentafluoroethyl)trifluorophosphate was obtained from Merck & Co. (Gibbstown N.J.). YPD is produced by Becton Dickinson (Sparks Md.).

### EXAMPLES 1-15

For Examples 1 through 15, a stock culture of Saccharomyces cerevisiae (W1362 20A) was grown by taking cells off an agar plate or from a frozen vial and placing them in a 15 mL sterile polypropylene test tube containing three milliliters of YPD medium. The test tube was incubated at 30° C. at 175 rpm in an Inova Incubator (Karlsbad, Sweden). After 24 hours the test tube was removed and stored at 4° C.

A portion (50 μL) of the stock culture prepared as above was inoculated into separate 15 mL sterile polypropylene culture tubes containing fresh YPD medium (3 mL). These tubes were incubated for four hours at 30° C. at 175 rpm. After four hours, one of these culture tubes was inoculated with 150 μL (5% v/v) of each of the ionic liquids as listed in Table 1 below. Each tube that was inoculated with an ionic liquid was also paired with a tube that received no ionic liquid and was used as a control.

All culture tubes were then incubated again under the same conditions for another 16 hours. After 16 hours, the culture tubes were stored at −80° C. Analytical samples were taken from each culture tube by thawing the samples and spinning them at 28,000 rpm and 20° C. for ten minutes in a Sorvall Instruments RC3C (Newtown Conn.) centrifuge. One milliliter of supernatant was removed from each tube and was subjected to high performance liquid chromatography (HPLC) analysis using an Agilent (Palo Alto Calif.) HPLC 1100 with a BioRad Aminex 87-H using 0.008 N sulfuric acid with both diode array and refractive index detection, and an Agilent HPLC 1100 with a Shodex OH-pak column using 0.01 N sulfuric acid. Data analysis was performed using the Agilent Chemstation software and Microsoft Excel.

The results are set forth in Table 2 as the Glucose Uptake Index (GUI) for both the examples and the controls. The glucose uptake index was calculated by taking the total glucose consumed in a fermentation sample, whether or not the sample contained an ionic liquid, and dividing it by the total initial glucose present in the broth at the start of fermentation.

$$GUI = \frac{\begin{matrix}( Media glucose ( mM ) - \\ Sample Final Glucose ( mM ) )\end{matrix}}{Media Glucose ( mM )} \times 100 \qquad Equation 1$$

The GUI for a sample varies directly with the extent of metabolic activity of the cells; and for the examples (in each of which an ionic liquid had been added to the broth), a relatively smaller or larger GUI may indicate the presence or absence, respectively, of an effect of the ionic liquid on metabolic activity.

TABLE 1

| IL cation | IL anion | IL cation | IL anion |
|---|---|---|---|
| Chemical Name | Chemical name | Abbreviation | Abbreviation |
| 1-hexyl-3-methylimidazolium | tris(pentafluoroethyl)trifluorophospate | [HMIM] | [FAP] |
| tetradecyl(tri-n- | 1,1,2-trifluoro-2- | [6,6,6,14-P] | [TPES] |
| hexyl)phosphonium | (perfluoroethoxy)ethanesulfonate | | |
| 1-butyl-3-methylimidazolium | tetrafluoroborate | [BMIM] | [BF₄] |
| 1-butyl-3-methylimidazolium | 1,1,2-trifluoro-2- | [BMIM] | [TPES] |
| | (perfluoroethoxy)ethanesulfonate | | |
| 1-ethyl-3-methylimidazolium | bis(pentafluoroethylsulfonyl)imide | [EMIM] | [BEI] |
| 1-ethyl-3-methylimidazolium | bis(trifluoromethylsulfonyl)imide | [EMIM] | [Tf₂N] |
| 1-butyl-3-methylimidazolium | hexafluorophosphate | [BMIM] | [PF₆] |
| 1-butyl-3-methylimidazolium | bis(trifluoromethylsulfonyl)imide | [BMIM] | [Tf₂N] |
| 1-hexyl-3-methylimidazolium | bis(trifluoromethylsulfonyl)imide | [HMIM] | [Tf₂N] |
| 1-butyl-3-methylimidazolium | 1,1,2-trifluoro-2- | [BMIM] | [TTES] |
| | (trifluoromethoxy)ethanesulfonate | | |
| 1-butyl-3-methylimidazolium | 1,1,2,3,3,3-hexafluoropropanesulfonate | [BMIM] | [HFPS] |
| 3-methyl-1-propylpyridinium | bis(trifluoromethylsulfonyl)imide | [PMPy] | [Tf₂N] |
| 1-butyl-3-methylimidazolium | 2-(1,2,2,2-tetrafluoroethoxy)-1,1,2,2- | [BMIM] | [FS] |
| | tetrafluoroethanesulfonate | | |
| 1-butyl-3-methylimidazolium | 1,1,2,3,3,3-hexafluoropropanesulfonate | [BMIM] | [HFPS] |
| 1-butyl-3-methylpyrrolidinium | bis(trifluoromethylsulfonyl)imide | [BMP] | [Tf₂N] |

TABLE 2

| Ex. | IL cation | IL anion | GUI of | GUI of |
|---|---|---|---|---|
| No. | Abbreviation | Abbreviation | Control^{b} | Example^{c} |
| 1 | [HMIM] | [FAP] | 94.6 | 99.7 |
| 2 | [6,6,6,14-P] | [TPES] | 94.6 | 78.4 |
| 3 | [BMIM] | [BF₄] | 94.6 | 13.3 |
| 4 | [BMIM] | [TPES] | 94.6 | 22.0 |
| 5 | [EMIM] | [BEI] | 94.6 | 100.0 |
| 6 | [EMIM] | [Tf₂N] | 94.6 | 76.8 |
| 7 | [BMIM] | [PF₆] | 94.6 | 48.2 |
| 8 | [BMIM] | [Tf₂N] | 75.1 | 97.6 |
| 9 | [HMIM] | [Tf₂N] | 75.1 | 89.5 |
| 10 | [BMIM] | [TTES] | 75.1 | 37.1 |
| 11 | [BMIM] | [HFPS] | 75.1 | 30.1 |
| 12 | [PMPy] | [Tf₂N] | 75.1 | 97.1 |
| 13 | [BMIM] | [FS] | 75.1 | 42.8 |
| 14 | [BMIM] | [HFPS] | 75.1 | 31.9 |
| 15 | [BMP] | [Tf₂N] | 100.0 | 100.0 |

^{b}GUI Control: Glucose Utilization Index in the absence of ionic liquid phase.
^{c}GUI Sample: Glucose Utilization Index in the presence of 5% (v/v) of the ionic liquid phase.

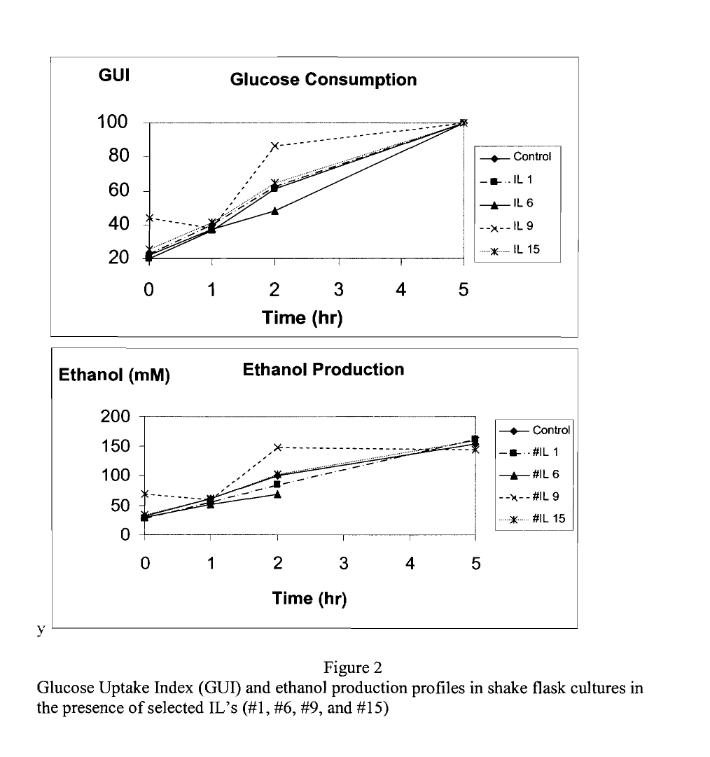

Scale-up experiments were performed using the same ionic liquids added to the fermentation broth in Examples 1, 6, 9 and 15. Cultures were again prepared using the same growth conditions as described above except that the total broth volume was 25 mL. The selected ionic liquids were added to a container of each broth in an amount of 5 volume percent. In these experiments, small samples were withdrawn from the broth for HPLC analysis periodically over a five hour time span to get a better idea of the extent of metabolic activity after ionic liquid addition as well as to provide information on glucose consumption and product formation. FIG. 2 shows a graphical representation of the HPLC data collected from the four scale-up runs and from a control in which no ionic liquid had been added to the broth. The calculation of GUI was made as described above, and amount of ethanol produced was taken directly from the HPLC data. As shown in FIG. 2, glucose uptake and ethanol production remained robust throughout the fermentation run for each sample.

### EXAMPLES 16-20

The extraction of ethanol and isobutanol from an aqueous solution by the ionic liquids used in Examples 1, 6, 9 and 15 was evaluated. 3 mL of an aqueous solution containing about 31 g/L of ethanol or isobutanol was mixed with 150 microliters (5 vol %) of the indicated ionic liquid in an airtight vial with minimal headspace. Ethanol and isobutanol concentration in the solution was measured using HPLC as described above following thorough mixing and overnight equilibration of the contents of the vial. HPLC results are shown in Table 3.

TABLE 3

| | | | Ethanol | | | Isobutanol | |
|---|---|---|---|---|---|---|---|
| | | | Remaining | | | Remaining | |
| | | | in | Ethanol | | in | Isobutanol |
| | | Ethanol | Solution | Extracted | Isobutanol | Solution | Extracted |
| Ex. | Ionic Liquid | (mM) | (g/L) | (%) | (mM) | (g/L) | (%) |
| 16 | Control | 684 | 31.5 | 0.00 | 416 | 30.9 | 0.00 |
| 17 | [HMIM] [FAP] | 680 | 31.3 | 0.45 | 404 | 29.9 | 3.14 |
| 18 | [EMIM] [Tf2N] | 670 | 30.9 | 1.96 | 389 | 28.8 | 7.11 |
| 19 | [HMIM] [Tf2N] | 678 | 31.3 | 0.76 | 388 | 28.8 | 7.28 |
| 20 | [BMP] [Tf2N] | 676 | 31.1 | 1.17 | 391 | 29.0 | 6.33 |

The amount of alcohol extracted to the ionic liquid phase was calculated as a percentage of the amount of alcohol remaining in the solution in the control (Example 16), which had no ionic liquid phase but was processed experimentally under the same conditions. In some cases, about 6-7% of isobutanol was extracted even though only 5% (V/V) of ionic liquid was used as the second phase.

What is claimed is:

1. A process for preparing butanol in a fermentation broth in a fermentor, comprising:
(a) providing a liquid fermentation broth that is comprised of a carbohydrate substrate, nutrients and water in which an alcohol is produced by the growth of Saccharomyces or a recombinant Saccharomyces;
(b) contacting at least one ionic liquid with the fermentation broth, or a portion thereof, to form from the resulting mixture an ionic liquid phase and an aqueous phase wherein the butanol, or a portion thereof, is more soluble in the ionic liquid phase than the aqueous phase; and
(c) separating the butanol-rich ionic liquid phase from the aqueous phase; and, optionally, recovering the butanol from the ionic liquid phase;
wherein the ionic liquid is comprised of (i) a cation selected from the group consisting of pyridinium, imidazolium and phosphonium, and (ii) an anion selected from the group consisting of tetrafluoroborate, hexafluorophosphate, 1,1,2,3,3,3-hexafluoropropanesulfonate, tris(pentafluoroethyl)trifluorophosphate, 1,1,2-trifluoro-2-(perfluoroethoxy)ethanesulfonate, 1,1,2-trifluoro-2-(perfluoromethoxy)ethanesulfonate, 2-(1,2,2,2-tetrafluoroethoxy)-1,1,2,2-tetrafluoroethanesulfonate, bis(pentafluoroethylsulfonyl)imide, and bis(trifluoromethylsulfonyl)imide.

2. A process according to claim 1 wherein, during or after separation of the ionic liquid phase from the aqueous phase, production of the butanol product in the fermentation broth continues.

3. A process according to claim 1 which is conducted in the fermentor.

4. A process according to claim 1 which is conducted in a vessel external to the fermentor.

5. A process according to claim 4 wherein, after separation of the aqueous phase from the ionic liquid phase, the aqueous phase or a portion thereof is returned to the fermentor.

6. A process according to claim 1 wherein, after butanol recovery, the ionic liquid is recycled to the fermentation broth.

7. A process according to claim 1 wherein an ionic liquid comprises a cation selected from the group consisting of 1-hexyl-3-methylimidazolium, tetradecyl(tri-n-hexyl)phosphonium, 1-butyl-3-methylimidazolium, 1-ethyl-3-methylimidazolium, 3-methyl-1-propylpyridinium, or 1-butyl-1-methylpyrrolidinium.

8. A process according to claim 1 wherein an ionic liquid is 1-hexyl-3-methylimidazolium tris(pentafluoroethyl)trifluorophosphate, tetradecyl(tri-n-hexyl)phosphonium 1,1,2-trifluoro-2-(perfluoroethoxy)ethanesulfonate, 1-butyl-3-methylimidazolium tetrafluoroborate, 1-butyl-3-methylimidazolium 1,1,2-trifluoro-2-(perfluoroethoxy)ethanesulfonate, 1-ethyl-3-methylimidazolium bis(pentafluoroethylsulfonyl)imide, 1-ethyl-3-methylimidazolium bis(trifluoromethylsulfonyl)imide, 1-butyl-3-methylimidazolium hexafluorophosphate, 1-butyl-3-methylimidazolium bis(trifluoromethylsulfonyl)imide, 1-hexyl-3-methylimidazolium bis(trifluoromethylsulfonyl)imide, 1-butyl-3-methylimidazolium 1,1,2-trifluoro-2-(trifluoromethoxy)ethanesulfonate, 1-butyl-3-methylimidazolium 1,1,2,3,3,3-hexafluoropropanesulfonate, 3-methyl-1-propylpyridinium bis(trifluoromethylsulfonyl)imide, 1-butyl-3-methylimidazolium 2-(1,2,2,2-tetrafluoroethoxy)-1,1,2,2-tetrafluoroethanesulfonate, 1-butyl-3-methylimidazolium 1,1,2,3,3,3-hexafluoropropanesulfonate, or 1-butyl-3-methylpyrrolidinium bis(trifluoromethylsulfonyl)imide.

9. A process according to claim 1 wherein at least one ionic liquid is 1-hexyl-3-methylimidazolium tris(pentafluoroethyl)trifluorophosphate, tetradecyl(tri-n-hexyl)phosphonium 1,1,2-trifluoro-2-(perfluoroethoxy)ethanesulfonate, 1-ethyl-3-methylimidazolium bis(pentafluoroethylsulfonyl)imide, 1-ethyl-3-methylimidazolium bis(trifluoromethylsulfonyl)imide, 1-butyl-3-methylimidazolium bis(trifluoromethylsulfonyl)imide, 1-hexyl-3-methylimidazolium bis(trifluoromethylsulfonyl)imide, 3-methyl-1-propylpyridinium bis(trifluoromethylsulfonyl)imide, or 1-butyl-3-methylpyrrolidinium bis(trifluoromethylsulfonyl)imide.

10. A process according to claim 1 further comprising admixing the recovered butanol with a motor fuel.
